(19) United States
(12) Patent Application Publication
(10) Pub. No.: US 2017/0136075 A1
(43) Pub. Date: May 18, 2017

(54) PHARMACEUTICAL COMPOSITION CONTAINING SPIRULINA MAXIMA EXTRACT AS ACTIVE INGREDIENT FOR PREVENTING AND TREATING RETINAL DISEASES

(71) Applicants: Woo Chang JUNG, Seoul (KR); JCREATION, Jeju-si Jeju-do (KR)

(72) Inventors: Se Young CHOUNG, Seoul (KR); Dong Joon KIM, Jeju-si Jeju-do (KR); Seung Won YANG, Yongin-si Gyeonggi-do (KR); Sang Ho CHOI, Suwon-si Gyeonggi-do (KR); Do Hyung KANG, Ansan-si Gyeonggi-do (KR); Soo Jin HEO, Ansan-si Gyeonggi-do (KR)

(73) Assignees: JCREATION, Jeju-si Jeju-do (KR); Woo Chang JUNG, Seoul (KR)

(21) Appl. No.: 15/376,424

(22) Filed: Dec. 12, 2016

Related U.S. Application Data
(63) Continuation of application No. PCT/KR2015/005952, filed on Jun. 12, 2015.

(30) Foreign Application Priority Data
Jun. 13, 2014 (KR) 10-2014-0071960

(51) Int. Cl. A61K 35/748; A23L 33/105; A61K 38/16

(52) U.S. Cl. CPC A61K 35/748; A61K 38/164; A23L 33/105; A23V 2002/00

(57) ABSTRACT

A Spirulina maxima extract of the present invention and Allophycocyanin (APC), R-phycoerythrin (R-PE), and C-phycocyanin (C-PC), which are components of the Spirulina maxima extract, show an effect of inhibiting cell death and A2E (pyridinium bis-retinoid), oxidation due to blue light, and therefore can be usefully applied as an active ingredient in a composition for preventing and treating retinal disease.

### FIELD OF THE INVENTION

The present invention relates to a composition containing Spirulina maxima extract as an active ingredient for preventing and treating retinal diseases.

### BACKGROUND OF THE INVENTION

In modern society, 80-90% of life information is obtained visually in the midst of overflowing visual culture, and the rate of decrease of eyesight is continuously increasing due to environmental reasons. While modern people spend most of their time in front of a variety of digital screens such as computers, smart phones, etc., because LEDs often being used on such smart device screens eyesight is being deterioriated by the blue light emitted therefrom. Blue light is the light with blue color detected in the 400˜500 nm range of visible light, emitted from digital devices or smart phones. A good example of a blue light emission is when a digital device is turned on at night, the light seems bluish overall even though it is white. If a person is exposed to this kind of blue light for a long time, the light stimulates the optic nerve to cause fatigue and other eye diseases.

Blue light has a strong energy and high penetrating power and therefore, once it passes through the eyes directly, it makes the retina lose focus, lowering visibility. Chronic exposure to blue light is a reason for aging and degeneration of the retina. Blue light's effect on human includes dry eye, eye fatigue, deterioration of eyesight, various eye diseases, accelerated aging of visual cells (retina), macular degeneration, and insomnia caused by melatonin synthesis inhibition (Ganka Gakkai Zasshi. 2001 October; 105(10):687-95; Archives of Ophthalmology 1992; 110:99-104; Review of Ophthalmology Oct. 15 2003; 10(10)).

The retina is a transparent thin layer of tissue which is located in the innermost part of the ocular wall and contacted with the vitreous body. It functions as the primary visual information system that converts optical information of an object into electrical signals and delivers the image through the optic nerve to the visual cortex of the brain. The retina comprises more than a hundred million light-sensitive photoreceptor cells, more than a million optic neurons or ganglion cells, and numerous neurons that connect those cells together. Therefore, the retina is the most sophisticated organ in the human body. The macula lutea resides at the center of the retina and distinguishes colors and objects and provides vision. The macula lutea forms a thinner part of retina that is composed of a layer of light-sensitive photoreceptor cells including cone cells and a layer of ganglion cells. In bright light, the electrical signals of images are converted into chemical signals which are delivered to the brain through the optic nerve or the neuron of the ganglion cells. The part of retina outside the macula lutea plays a rold in recognition of the periphery and also provides vision in the dark. Approximately 30% of our brain cells are used to process the visual information sent by the retina. Once problems occur in the retina due to either aging or external factors, it leads to visual impairment that gradually deteriorates visual acuity and visual field until finally reach to blindness. Retinal disease is mainly divided into three groups. First, retinal detachment is caused by abnormal retinal peripheral tissues. When the neural retina is detached from the retinal pigment epithelium then the retina layer is separated to the backside of the eyeball, causing visual impairment. Second, peripheral retinal degeneration can be caused by abnormalities in the retinal peripheral tissues. Third, macular degeneration can be caused by problems in the macula lutea. Once the retina is detached from the retinal pigment epithelium, it cannot receive optical information of the image and neurons cannot function properly by failure of getting nutrition supply from the choroid. If this problem is neglected, permanent neurodeatrophia is caused, resulting in blindness. Visual impairment resulting from retinal disease is the main cause of blindness developed with aging. It can be also caused by genetic reasons, high myopia, trauma and the like. Blindness is the second most frequent ophthalmic disease after cataract. Three major ophthalmic diseases causing blindness are diabetic retinopathy, macular degeneration, and glaucoma. Retinal disease is not lethal. However, along with the increasing senior population, industrialization and change in diet, retinal disease has recently been rapidly increasing. Therefore, there is a need to develop a therapeutical composition for treating a retinal disease that can be administered in the form of traiditonal natural medicine other than relying on synthetic drugs or surgical methods.

Spirulina maxima is a kind of microalgae that are reproduced in salty alkali tropical area, for example in Lake Chad, Africa, and in Lake Texcoco, Mexico. Spirulina maxima cell contains a large volume of chlorophyll and phycocyanin, by which it absorbs the sun light in order to actively assimilate carbon dioxide to grow. Due to such pigments, the algae is blue-green and therefore it has been classified as blue-green algae.

Since electron microscope was developed, the cellular structure of microorganism has been accurately identified. As a result, it was disclosed that the structure of green algae or brown algae was different from the structure of higher plants. That is, the structure of green algae is a eukaryote structure, which is equal to that of a higher plant. On the other hand, since the early 1960s, blue-green algae was identified to have a prokaryote structure which was similar to the structure of bacteria, therefore, some microbiologists have made statements that blue-green algae is closer to bacteria than to algae and needs to be classified as a Bacteriomycota. Today, this statement is accepted and therefore the blue-green algae above is now classified into the group of blue-green bacteria. However, in the industrial field, it is still conventionally called “micro-algae.”

The name Spirulina maxima originated from its spiral shape. Having double-helix DNA and primitive structures, it is a spiral bacterium called cyanobacteria that has characteristics between animal and plant. Spirulina maxima is an edible microorganism composed of 55˜70% proteins, 6-9% lipids, 15˜20% carbohydrates, and minerals, vitamins, fibers, and figments. Spirulina maxima is not only high in protein but also includes all 8 essential amino acids. The lipids found in this microorganism are free-fatty acids, and 70-80% of which are linoleic acid, γ-linolenic acid etc. Spirulina maxima has a low concentration of carbohydrates. However, it contains rhamnose and glycogen that can be absorbed without help of insulin, thereby being useful as an energy source for diabetes patients. Native people have traditionally consumed this micro-algae for food for a long time. Nutritional studies confirmed that the nutritional composition of this alge with high concentration of proteins and other nutrients including amino acids is very beneficial for human health. It is known that the beneficial ingredients mentioned above include Allophycocyanin, R-phycoerythin, and C-phycocyanin (Nanni B. et al., Microbiol Res. 2001; 156(3):259-66; Hangeul Donguibogam http://donguibogam.co.kr).

The present invention relates to developing a composition for preventing and treating retinal disease. The inventors demonstrated that Spirulina maxima extract had the effect of inhibiting oxidized A2E (pyridinium bis-retinoid) and cell death caused by blue light, thereby confirmed that said Spirulina maxima extract and the components of the same, Allophycocyanin, R-phycoerthin, and C-phycocyanin could be used as a therapeutic composition useful for preventing and treating retinal disease.

### DETAILED DESCRIPTION OF THE INVENTION

### Technical Task

The purpose of the present invention is to provide a pharmaceutical composition for preventing and treating retinal disease and a health functional food for improving conditions of retinal disease comprising Spirulina maxima extract and Allophycocyanin, R-phycoerythrin, or C-phycocyanin as an active ingredient.

### Technical Solution

In order to achieve this goal the present invention provides a pharmaceutical composition for preventing and treating retinal disease comprising Spirulina maxima extract as an active ingredient.

The present invention also provides a health functional food for preventing and improving conditions of retinal disease comprising Spirulina maxima extract as an active ingredient.

The present invention further provides a pharmaceutical composition for preventing and treating retinal disease comprising Allophycocyanin, R-phycoerythrin, or C-phycocyanin as an active ingredient.

In addition, the present invention provides a health functional food for preventing and improving conditions of retinal disease comprising Allophycocyanin, R-phycoerythrin, or C-phycocyanin as an active ingredient.

### Advantageous Effect

The Spirulina maxima extract of the present invention and Allophycocyanin (APC), R-phycoerythrin (R-PE), and C-phycocyanin (C-PC), which are the components of the Spirulina maxima extract, show an effect of inhibiting the oxidation of A2E (pyridinium bis-retinoid) and cell death caused by blue light, so that the present invention can be efficiently applied as an active ingredient in a composition for preventing and treating retinal disease.

### BRIEF DESCRIPTION OF THE DRAWINGS

FIG. 1 is a graph illustrating the inhibiting effect of Allophycocyanin (APC), R-phycoerythrin (R-PE), and C-phycocyanin (C-PC) against oxidized A2E (pyridinium bis-retinoid).

FIG. 2 is a graph illustrating the cell protecting effect of Allophycocyanin, R-phycoerythrin, and C-phycocyanin against cell death caused by blue light in ARPE-19 cells accumulated with A2E (post-treated with the sample).

A2E: the cells not treated with blue light after A2E accumulation,

A2E BL: A2E the cells treated with blue light after A2E accumulation (negative control),

APC: 25 μg/ml,

R-PE: 25 μg/ml, and

C-PC: 6.25, 12.5, 25, 50, and 100 μg/ml.

FIG. 3a is a graph illustrating the cell protecting effect of Allophycocyanin, R-phycoerythrin, and C-phycocyanin against cell death caused by blue light in ARPE-19 cells (preliminary experiment for FIG. 3b).

APC: 12.5, 25, and 40 μg/ml,

R-PE: 12.5, 25, and 40 μg/ml, and

C-PC: 12.5, 25, 50, 100, and 200 μg/ml.

FIG. 3b is a graph illustrating the cell protecting effect of Allophycocyanin, R-phycoerythrin, and C-phycocyanin against cell death caused by blue light in ARPE-19 cells (pre-treated with the sample).

APC: 12.5 μg/ml,

R-PE: 12.5 μg/ml, and

C-PC: 25, 50, and 100 μg/ml.

FIG. 4 is a graph illustrating the cell protecting effect of Spirulina maxima extract (products in Myanmar, Hi., and KIOST) against cell death in ARPE-19 cells accumulated with A2E (post-treated with the sample).

FIG. 5a is a graph illustrating the cell protecting effect of Spirulina maxima extracts (products in Myanmar, Hi., and KIOST) against cell death in ARPE-19 cells (preliminary experiment for FIG. 5b).

product in Myanmar: 125, 250, 500, 750, and 1000 μg/ml,

product in Hawaii: 250, 500, and 1000 μg/ml, and

product in KIOST: 250, 500, and 1000 μg/ml.

FIG. 5b is a graph illustrating the cell protecting effect of Spirulina maxima extract (product in KIOST) against cell death caused by blue light in ARPE-19 cells (pre-treated with the sample).

product in KIOST: 62.5, 125, 250, and 500 μg/ml.

### DESCRIPTION OF THE PREFERRED EMBODIMENTS

Hereinafter, the present invention is described in detail.

The present invention provides a pharmaceutical composition for preventing and treating retinal disease comprising Spirulina maxima extract as an active ingredient.

The said Spirulina maxima extract preferably contains one or more ingredients selected from the group consisting of Allophycocyanin (APC), R-phycoerythrin (R-PE), and C-phycocyanin (C-PC).

The Spirulina maxima extract is preferably, but not limited thereto prepared by the method comprising the following steps:

1) extracting Spirulina maxima after adding an extraction solvent to Spirulina maxima;

2) filtering the extract prepared in step 1); and

3) concentrating the extract filtered in step 2) under reduced pressure, followed by drying thereof.

In the method above, the Spirulina maxima of step 1) may be either cultivated or purchased.

The extraction solvent herein is preferably water, alcohol, or the mixture thereof. The said alcohol is preferably C₁˜C₂ lower alcohol. The lower alcohol herein is preferably ethanol or methanol. The extraction method is preferably high temperature extraction under reduced pressure, boiling extraction, reflux extraction, hot-water extraction, enfleurage, room temperature extraction, ultrasonic extraction, centrifugal extraction, or vapor extraction, but not always limited thereto. The extraction solvent is preferably added to Spirulina maxima at the ratio of 1˜10 times the Spirulina maxima volume. The preferable temperature for the extraction is 30° C.˜100° C., but not always limited thereto. The extraction hours are preferably 2˜48 hours, but not always limited thereto. The extraction is preferably repeated 2˜5 times, but not always limited thereto.

In this method, concentration under reduced pressure in step 3) is preferably performed by using a vacuum concentrator or a vacuum rotary evaporator, but not always limited thereto. Drying herein is preferably performed by reduced-pressurized drying, vacuum drying, boiling drying, spray drying, or freeze drying, but not always limited thereto.

The above Spirulina maxima extract has the effect of inhibiting A2E (pyridinium bis-retinoid) oxidized by blue light and can inhibit retinal cell death.

The retinal disease herein is one or more diseases selected from the group consisting of macular degeneration, glaucoma, Usher syndrome, Stargardt disease, Bardet-Biedl syndrome, Best disease, choroideremia, gyrate-atrophy, retinitis pigmentosa, macular degeneration, Leber congenital amaurosis (Leber's Hereditary Optic Neuropathy), BCM (blue-cone monochromacy), retinoschisis, ML (Malattia Leventinese), Oguchi disease, and Refsum disease.

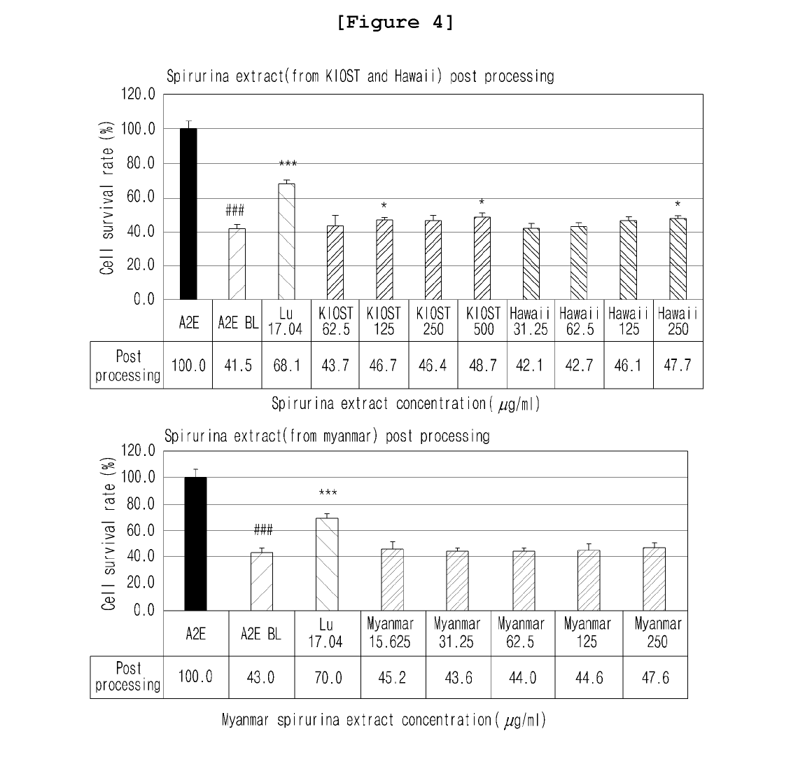

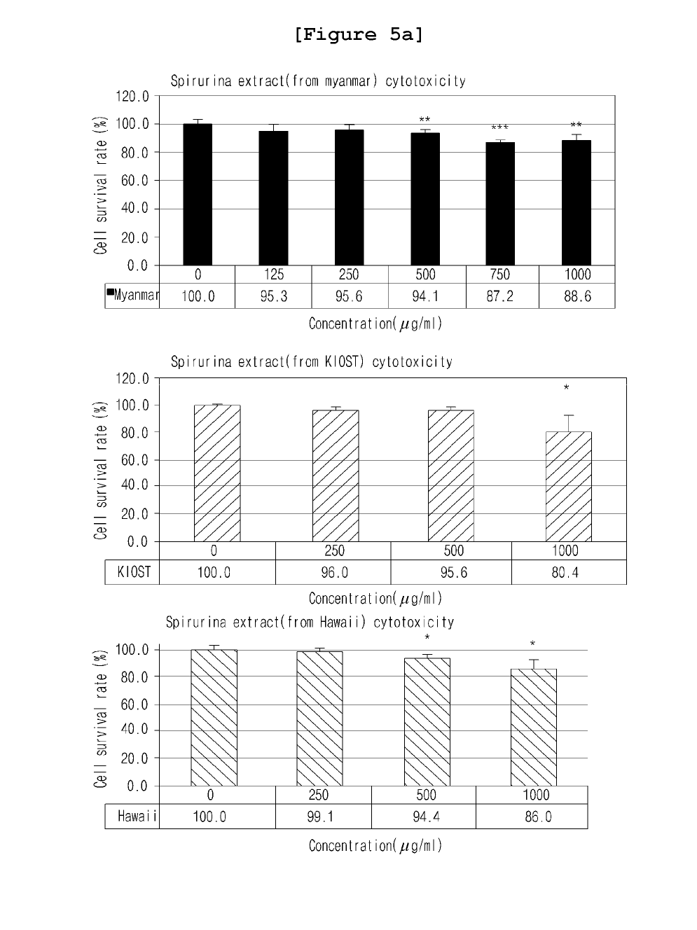

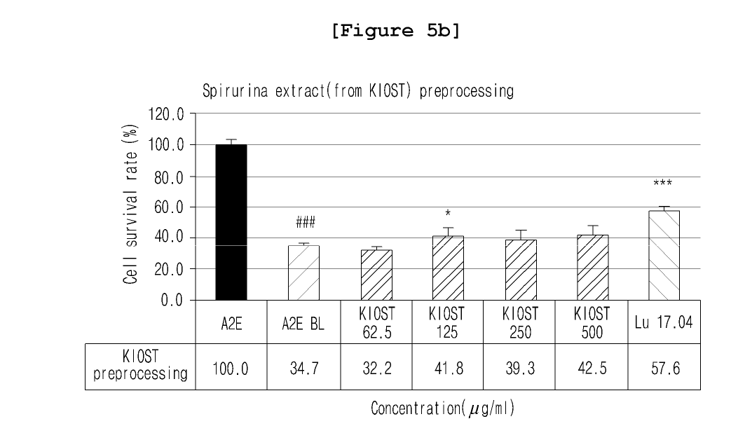

In a preferred embodiment of the present invention, the cell protecting effect of Spirulina maxima extracts (products in Myanmar, Hi., and KIOST) from retinal cell death caused by blue light in ARPE-19 cells containing A2E accumulated therein was measured. As a result, the Spirulina maxima extract from the Spirulina maxima originated from Hawaii and KIOST exhibited the cell protecting effect dose-dependently. However, the Spirulina maxima extract from Myanmar orgin did not display statistically significant cell protecting effect (see FIG. 4). Further, based on the KIOST originated Spirulina maxima extact dose related to retinal cell death in ARPE-19 cells, obtained in FIG. 5a, the cell protecting effect based on A2E accumulation and the inhibition of photooxidation was measured. As a result, survival rate (%) of the cells treated with the KIOST originated Spirulina maxima extract at the concentrations of 62.5, 125, 250, and 500 μg/ml was increased respectively by 0%, 10.8%, 7.0%, and 11.9% (see FIG. 5b).

Therefore, the Spirulina maxima extract (products in Hawaii and KIOST) had the cell protecting effect from cell death caused by blue light and had excellent cell protecting effect when photooxidation was inhibited, suggesting that the Spirulina maxima extract could be used as a pharmaceutical composition for preventing and treating retinal disease.

The pharmaceutical composition containing the extract of the present invention can include, in addition to the extract, one or more effective ingredients having the same or similar function to the extract.

The pharmaceutical composition of the present invention can additionally include a pharmaceutically acceptable additive, which is exemplified by starch, gelatinized starch, microcrystalline cellulose, lactose, povidone, colloidal silicon dioxide, calcium hydrogen phosphate, lactose, mannitol, taffy, Arabia rubber, pregelatinized starch, corn starch, cellulose powder, hydroxypropyl cellulose, Opadry, sodium carboxy methyl starch, carunauba wax, synthetic aluminum silicate, stearic acid, magnesium stearate, aluminum stearate, calcium stearate, white sugar, dextrose, sorbitol, talc, etc. The pharmaceutically acceptable additive herein is preferably added by 0.1-90% of the weight of the pharmaceutical composition.

Therefore, the pharmaceutical composition of the present invention can be administered orally or parenterally and be used in general forms of pharmaceutical formulation. The composition of the present invention may be prepared for oral or parenteral administration by mixing with generally used diluents or excipients such as fillers, extenders, binders, wetting agents, disintegrating agents and surfactant. Solid formulations for oral administration include tablets, pills, powders, granules and capsules. These solid formulations may be prepared by mixing the extract of the invention with one or more excipients such as starch, calcium carbonate, sucrose, lactose, or gelatin, etc. Also, instead of excipients, a lubricant such as magnesium stearate talc can be used. Liquid formulations for oral administrations include suspensions, solutions, emulsions and syrups, etc. and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations and suppositories. For water insoluble excipients and suspensions, propylene glycol, polyethylene glycol, vegetable oil like olive oil, injectable ester like ethylolate, etc. can be used. For suppositories, witepsol, macrogol, tween 61, cacao butter, laurin butter, glycerogelatin, etc. can be used.

The pharmaceutical composition of the present invention can be administered orally or parenterally and for parenteral administration it is preferable to use skin external application, intraperitoneal injection, subcutaneous injection, intravenous injection, intramuscular injection or intrathoracic injection. The dosage can vary according to weight, age, gender, health condition, diet, administration frequency, administration method, excretion rate and severity of disease of patient.

The dosage of the composition of the present invention can vary according to weight, age, gender, health condition, diet, administration frequency, administration method, excretion rate and severity of disease of the patient. The daily dose based on amount of extract is 0.0001˜100 mg/kg, and preferably 0.001˜10 mg/kg, and administration frequency can be 1˜6 times a day.

The pharmaceutical composition of the present invention can be administered alone or together with surgical operation, hormone therapy, chemo-therapy and biological regulators to prevent and treat retinal disease. The present invention provides a pharmaceutical composition for preventing and treating retinal disease comprising one of the components selected from the group consisting of Allophycocyanin, R-phycoerythrin, and C-phycocyanin as an active ingredient.

The above-mentioned Allophycocyanin, R-phycoerythrin, or C-phycocyanin has the effect of inhibiting A2E (pyridinium bis-retinoid) oxidized by blue light and can inhibit retinal cell death.

The retinal disease herein is one or more diseases selected from the group consisting of macular degeneration, glaucoma, Usher syndrome, Stargardt disease, Bardet-Biedl syndrome, Best disease, choroideremia, gyrate-atrophy, retinitis pigmentosa, macular degeneration, Leber congenital amaurosis (Leber's Hereditary Optic Neuropathy), BCM (blue-cone monochromacy), retinoschisis, ML (Malattia Leventinese), Oguchi disease, and Refsum disease.

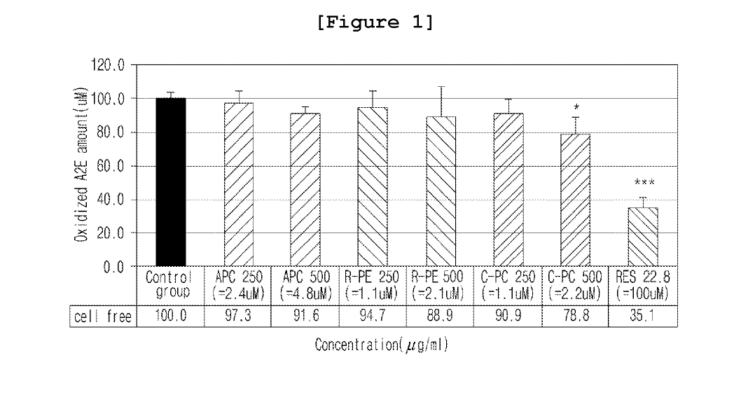

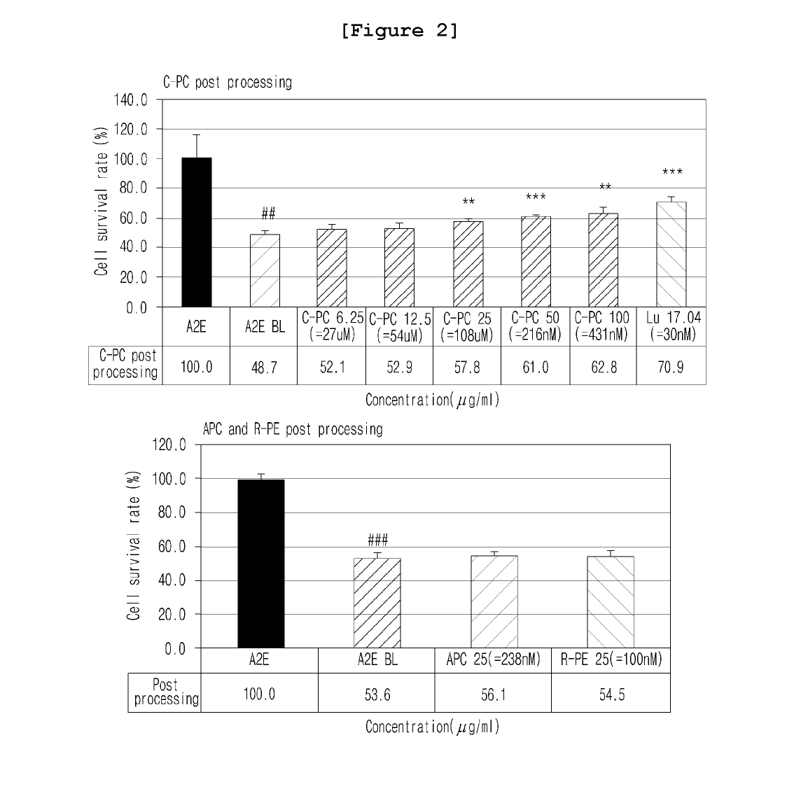

In a preferred embodiment of the present invention, the inventors measured the oxidation inhibiting effect of Allophycocyanin (APC), R-phycoerythrin (R-PE), and C-phycocyanin (C-PC) on pyridinium bis-retinoid (A2E) oxidized by blue light. As a result, the oxidation inhibiting effect of C-PC on A2E was statistically significant. In particular, the oxidation inhibiting effect of C-PC on A2E was the greatest, followed by R-PE and APC in that order (C-PC>R-PE>APC) (see FIG. 1). The cell protecting effect of APC, R-PE, and C-PC against retinal cell death was investigated in ARPE-19 cells wherein A2E was accumulated. As a result, in the group treated with C-PC, the cell survival rate was increased dose-dependently by 6.7%, 8.1%, 17.8%, 23.9%, and 27.6%, while the cytotoxicity was not observed in the groups treated with APC and R-PE (see FIG. 2).

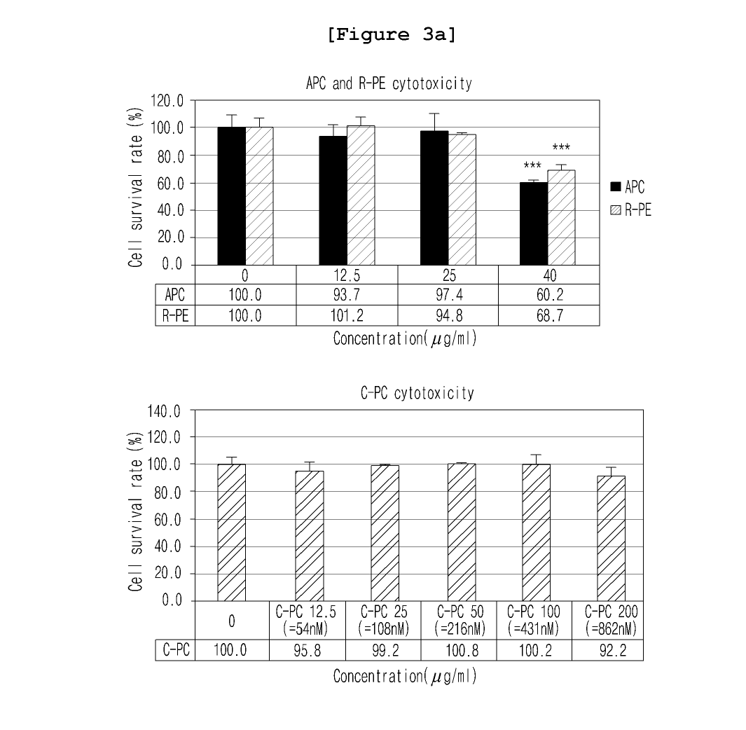

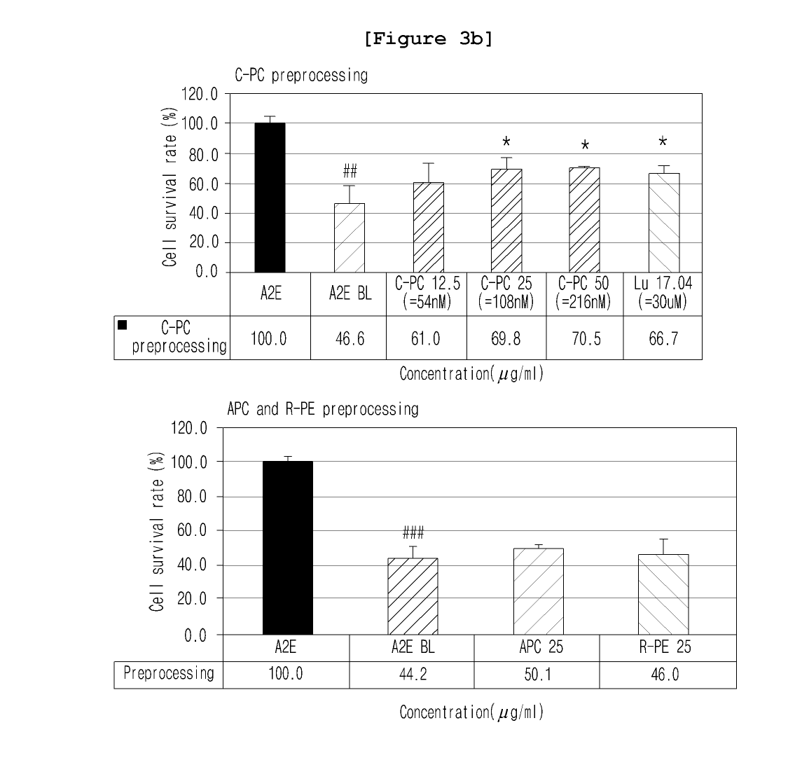

ARPE-19 cells were pre-treated with APC (25 μg/ml), R-PE (25 μg/ml), and C-PC (6.25, 12.5, 25, and 50 μg/ml) according to the selected C-PC concentrations in FIG. 3a. After confirming the accumulation of A2E, the cells were irradiated with blue light and cell survival rate was investigated. In the group treated with C-PC, the cell survival rate was increased dose-dependently. However, in those groups treated with APC and R-PE, the cell protecting effect was not statistically significant (see FIG. 3b).

Therefore, APC, R-PE, or C-PC was confirmed to have cell protecting effect against cell death caused by blue light and was excellent in cell protection based on inhibition of photooxidation, so that APC, R-PE, or C-PC can be advantageously used as a pharmaceutical composition for preventing and treating retinal disease.

The present invention also provides a heath functional food for preventing and improving conditions of retinal disease comprising Spirulina maxima extract as an active ingredient.

The said Spirulina maxima extract has the effect of inhibiting A2E (pyridinium bis-retinoid) oxidized by blue light and can inhibit retinal cell death.

The retinal disease herein is preferably one or more diseases selected from the group consisting of macular degeneration, glaucoma, Usher syndrome, Stargardt disease, Bardet-Biedl syndrome, Best disease, choroideremia, gyrate-atrophy, retinitis pigmentosa, macular degeneration, Leber congenital amaurosis (Leber's Hereditary Optic Neuropathy), BCM (blue-cone monochromacy), retinoschisis, ML (Malattia Leventinese), Oguchi disease, and Refsum disease.

Therefore, the Spirulina maxima extract has cell protecting effect against cell death caused by blue light and is excellent in cell protection based on inhibition of photooxidation, so that the Spirulina maxima extract can be advantageously used as a heath functional food for preventing and improving conditions of retinal disease.

In addition, the present invention provides a method for treating or preventing retinal disease containing the step of administering an effective dose of Spirulina maxima extract to a subject having retinal disease or a normal subject.

The present invention also provides a Spirulina maxima extract for the drug for preventing and treating retinal disease or for the health functional food for preventing and improving conditions of retinal disease.

The present invention also provides a health functional food for preventing and improving conditions of retinal disease comprising at least one or two of those active ingredients selected from the group consisting of Allophycocyanin, R-phycoerythrin, and C-phycocyanin.

The said Allophycocyanin, R-phycoerythrin, or C-phycocyanin has the effect of inhibiting A2E (pyridinium bis-retinoid) oxidized by blue light and inhibits retinal cell death.

Therefore, APC, R-PE, or C-PC was confirmed to have cell protecting effect against cell death caused by blue light and was excellent in cell protection based on inhibition of photooxidation, so that APC, R-PE, or C-PC can be advantageously used as a health functional food for preventing and improving conditions of retinal disease.

In addition, the present invention provides a method for treating or preventing retinal disease containing the step of administering an effective dose of at least one or two of those ingredients selected from the group consisting of Allophycocyanin, R-phycoerythrin, and C-phycocyanin to a subject having retinal disease or a normal subject.

The present invention also provides at least one or two of those active ingredients selected from the group consisting of Allophycocyanin, R-phycoerythrin, and C-phycocyanin for the drug for preventing and treating retinal disease or for the health food for preventing and improving conditions of retinal disease.

Practical and presently preferred embodiments of the present invention are illustrated in detail in the following Examples.

However, the present invention is not limited to the practical and presently preferred embodiments as those are examples only.

### Example 1: Preparation of Spirulina maxima Extract

Spirulina maxima (Korea Marine Microalgae Culture Center Accession No: KMMCC-1057) was provided from Korea Marine Microalgae Culture Center, Department of Marine Bio-materials & Aquaculture, Pukyong National University, Korea.

Particularly, the cultured Spirulina maxima was centrifuged in multi-tube carrier refrigerated centrifuge (Vision Scientific CO. Ltd) at 3000 rpm for 25 minutes. The separated cells were washed simply with 1.0% NaCl solution, followed by centrifugation again. The obtained cells were freeze-dried, which were used as a sample for phycocyanin extraction. The extraction was performed as follows: 10 ml of 0.1 M phosphate buffer (pH 7.0) was added to 40 mg of the freeze-dried sample, followed by vortexing for 15 minutes. Supernatant was obtained by centrifugation (3,500 rpm, 5 minutes), which was used as a Spirulina maxima extract.

### Example 2: Preparation of Allophycocyanin, R-phycoerythrin, or C-phycocyanin

Allophycocyanin (A7472), R-phycoerythrin (P6161), and C-phycocyanin were purchased from Sigma-Aldrich, which were prepared at the concentrations of 4 mg/ml, 10 mg/ml, and 1 mg/ml, respectively.

### Example 3: Cell Culture

Human adult ARPE cells (ARPE-19: catalog no. CRL-2302) used for the experiment and analysis in this invention were distributed from Vision Science Research Center, College of Medicine, The Catholic University of Korea. The ARPE cells above were cultured in DMEM supplemented with 10% FBS, 100 U/ml of penicillin, and 100 mg/ml of streptomycin in a 5% CO₂, 37° C. incubator. The cells were inoculated in a 6-well plate at the density of 5×10⁴ cells/well for the experiment.

### Example 4: A2E Synthesis Method

A2E (pyridinium bis-retinoid) used for the experiment and analysis in this invention was synthesized as follows: all-trans-retinal dissolved in ethanol was mixed with ethanol amine (molar ratio: 2:1), acetic acid was added to the mixture in the dark room, followed by reaction for 2 days. Then, the mixture was synthesized by being vacuum-concentrated at 40° C., followed by purification using silica gel column chromatography. The synthesized A2E was dissolved in DMSO (Dimethyl Sulfoxide) at the stock concentration of 20 mM, which was stored at −20° C. until the experiment.

### Experimental Example 1: Oxidation Inhibition Effect of Allophycocyanin, R-Phycoerythrin, and C-Phycocyanin on Pyridinium Bis-Retinoid (A2E) Oxidized by Blue Light

The following experiment was performed to measure the oxidation inhibition effect of Allophycocyanin (APC), R-phycoerythrin (R-PE), and C-phycocyanin (C-PC) due to retinal A2E oxidation.

Particularly, 20 μl of A2E (final concentration: 100 uM) was dissolved in 160 μl of PBS containing 0.01% DMSO, and the mixed solution was distributed in a 96-well (180 μl/well). Each sample (control or APC, R-PE, and C-PC obtained from Spirulina maxima extract) was added (respectively 250 and 500 μg/ml at the final concentration) to the plate (20 μl/well). Optical Density (OD), OD₄₃₀ (430 nm: A2E absorption wavelength), was measured with ELISA microplate reader. The plate was irradiated with blue light for 10 minutes at the energy strength of 2.01 J/cm², followed by measurement of OD again by the same manner as described above. The measured OD value was converted into the concentration by using A2E standard curve and the concentration of the oxidized A2E was calculated by the difference in concentration before and after the blue light irradiation.

As a result, as shown in FIG. 1, when C-PC was treated to the cells (250 and 500 μg/ml), the oxidized A2E was reduced by 9.1% and 21.2% compared to the control (CTL, 100%), from which it was confirmed that C-PC had a statistically significant A2E oxidation inhibition effect dose-dependently. In the meantime, when APC was treated to the cells (250 and 500 μg/ml), the oxidized A2E was reduced by 2.7% and 8.4% compared to the control. When R-PE was treated to the cells (250 and 500 μg/ml, the oxidized A2E was reduced by 5.3% and 11.1% compared to the control. Therefore, it was confirmed that APC and R-PE both had the A2E oxidation inhibition effect, which was though not as great as that of C-PC. So, the A2E oxidation inhibition effect was decreased in the following order: C-PC>R-PE>APC (FIG. 1).

### Experimental Example 2: Experiment for Cell Protection Effect of APC, R-PE, and C-PC Against Retinal Cell Death in ARPE-19 Cells Having A2E Accumulated Therein (Sample Post-Treatment System)

The following experiment was performed to investigate the cell protection effect of Allophycocyanin (APC), R-phycoerythrin (R-PE), and C-phycocyanin (C-PC) upon retinal A2E oxidation.

Particularly, ARPE-19 cells were distributed in a 24-well plate at the density of 2×10⁴ cells/well, followed by accumulation of A2E (20 μl) for 7 days (final concentration: 10 uM, three times of treatment). Thereafter, according to the result of FIG. 1, those three substances, APC (25 μg/ml), R-PE (25 μg/ml), and C-PC (6.25, 12.5, 25, 50, and 100 μg/ml were treated to the cells twice for 3 days. The cells were irradiated with blue light (4.02 J/cm²), followed by culture for 24 hours. Cell survival rate was measured by MTT assay. MTT assay is established based on the principal that yellow tetrazolium salt (MTT) reacts to reductase in mitochondria in a living cell to form purple formazan crystals. That is, as the population of live cells increases, the production of formazan crystals increases, resulting in the increased OD.

In MTT assay, DMEM containing 0.5 mg/ml of MTT was added, and light was blocked, followed by culture in a 37° C. incubator for 4 hours. Upon completion of the reaction, the cells were fully dissolved in 1 ml of DMSO. OD₅₄₀ was measured with ELISA microplate reader. The cell survival rate was presented with % by the cell survival rate of the cell group (normal control; A2E) that had A2E accumulated but not irradiated with blue light.

As a result, as shown in FIG. 2, there was a significant difference between the cell group having A2E accumulated but not irradiated with blue light (A2E) and the cell group having A2E accumulated and irradiated with blue light (negative control: A2E BL). Compared with the cell survival rate of the negative control A2E (100%), the cell survival rate of cells treated with those three substances respectively (APC: 25 μg/ml, R-PE: 25 μg/ml, C-PC: 6.25, 12.5, 25, 50, and 100 μg/ml) was increased as high as 6.7%, 8.1%, 17.8%, 23.9%, and 27.6% in the group that was treated with C-PC. On the other hand, in those groups treated with APC and R-PE at the concentration of 25 μg/ml, which was the highest concentration that was free from cytotoxicity, the cell protection effect was not significant statistically (FIG. 2).

### Experimental Example 3: Cell Protection Effect of Allophycocyanin, R-Phycoerythrin, and C-Phycocyanin from Retinal Cell Death Based on A2E Accumulation and the Inhibition of Photooxidation (Sample Pre-Treatment System)

The following experiment was performed to investigate A2E accumulation and cell protection effect of Allophycocyanin (APC), R-phycoerythrin (R-PE), and C-phycocyanin (C-PC) due to retinal A2E oxidation.

In the preliminary experiment (FIG. 3a), the cytotoxicity caused by 0˜40 μg/ml of APC or R-PE and 0˜200 μg/ml of C-PC was investigated and the concentrations displaying the effect on cytotoxicity were selected.

Particularly, ARPE-19 cells were distributed in a 24-well plate at the density of 2×10⁴ cells/well, which were treated with the three substances (final conc., APC: 25 μg/ml, R-PE: 25 μg/ml, C-PC: 6.25, 12.5, 25, and 50 μg/ml) on day 1, on day 4, and on day 7, three times in total. To accumulate A2E in cells, the cells were treated with A2E at the final concentration of 10 uM on day 2, on day 5, and on day 8, three times for 7 days, which were then irradiated with blue light (4.02 J/cm²), followed by culture for 24 hours. Then, the cell survival rate was measured by MTT assay. According to MTT assay method, DMEM containing 0.5 mg/ml of MTT was added to the cells, and light was blocked, followed by reaction in a 37° C. incubator for 4 hours. Upon completion of the reaction, the cells were fully dissolved in 1 ml of DMSO. Then, OD₅₄₀ was measured with ELISA microplate reader. Cell survival rate was calculated by comparing that of the cell group having A2E accumulated but not irradiated with blue light and presented as % by that.

As a result, as shown in FIG. 3b, there was a statistically significant difference between the group having A2E accumulated and not-irradiated with blue light (A2E) and the group having A2E accumulated and irradiated with blue light (negative control: A2E BL). The cell survival rate of the group treated with C-PC at the concentrations of 12.5, 25, and 50 μg/ml was increased 26.9%, 43.4%, and 44.8% respectively compared to the cell survival rate of the negative control A2E BL. In the meantime, in the groups treated with APC and R-PE, the cell protection effect was not so significant at the concentration of 25 μg/ml that was the highest concentration not causing cytotoxicity (FIG. 3b).

### Experimental Example 4: Cell Protection Effect of Spirulina maxima Extract According to the Origins on Retinal Cell Death in ARPE-19 Cells Having A2E Accumulated (Sample Post-Treatment System)

The following experiment was performed to investigate cell protection effect of Spirulina maxima extract with different origins (Myanmar, Hi., and KIOST) due to retinal A2E oxidation.

Particularly, ARPE-19 cells were distributed in a 24-well plate at the density of 2×10⁴ cells/well, and A2E was accumulated therein for 7 days by the same manner as described in Experimental Example 3 (final conc., 10 uM, three times). Then, the cells were treated with the three substances having different origins as follows: treated with the Myanmar originated Spirulina maxima extract at the final concentrations of 15.625, 31.25, 62.5, 125, and 250 μg/ml, with the Hawaii originated Spirulina maxima extract at the final concentrations of 31.25, 62.5, 125, and 250 μg/ml, and with the KIOST originated Spirulina maxima extract at the final concentrations of 62.5, 125, 250, and 10 μg/ml, followed by irradiation with blue light (4.02 J/cm²). The cells were cultured for 24 hours. According to MTT assay method, DMEM containing 0.5 mg/ml of MTT was added to the cells, and light was blocked, followed by reaction in a 37° C. incubator for 4 hours. Upon completion of the reaction, the cells were fully dissolved in 1 ml of DMSO, and OD₅₄₀ was measured with ELISA microplate reader, followed by presenting the cell survival rate as a percent(%)value calculated from the ratio of the survived cells to the number of cells having A2E accumulated but not irradiated with blue light.

As a result, as shown in FIG. 4, there was a statistically significant difference in the cell survival rate between the group having A2E accumulated but not irradiated with blue light (A2E) and the group having A2E accumulated and irradiated with blue light (negative control: A2E BL). The cell survival rate of each group treated respectively with three different extracts having different origins, precisely treated with the Myanmar originated Spirulina maxima extract at the concentrations of 15.625, 31.25, 62.5, 125, and 250 μg/ml, with the Hawaii originated Spirulina maxima extract at the concentrations of 31.25, 62.5, 125, and 250 μg/ml, and with the KIOST originated Spirulina maxima extract at the concentrations of 62.5, 125, 250, and 500 μg/ml, was calculated based on the cell survival rate (100%) of the negative control. As a result, the cell survival rate of the group treated with the Hawaii originated Spirulina maxima extract was increased 1.0%, 2.0%, 7.9%, and 10.6%, respectively. In the meantime, the cell survival rate of the group treated with the KIOST originated Spirulina maxima extract was increased 3.8%, 8.9%, 8.4%, and 12.4%, respectively. Therefore, it was confirmed that the cell protection effect of the Hawaii originated Spirulina maxima extract was almost equal to that of the KIOST originated Spirulina maxima extract. On the other hand, in the group treated with the Myanmar originated Spirulina maxima extract, the cell protection effect was not statistically significant at any concentration (FIG. 4).

### Experimental Example 5: Cell Protection Effect of KIOST Originated Spirulina maxima Extract on Retinal Cell Death in ARPE-19 Cells Based on A2E Accumulation and the Inhibition of Photooxidation (Sample Pre-Treatment System)

The following experiment was performed to investigate A2E accumulation and cell protection effect of the KIOST originated Spirulina maxima extract on due to retinal A2E oxidation.

In the preliminary experiment (FIG. 5a), the concentration that exhibited effect on cytotoxicity was selected after treating cells with 1˜1000 μg/ml of the KIOST originated Spirulina maxima extract. Particularly, ARPE-19 cells were distributed in a 24-well plate at the density of 2×10⁴ cell/well. The cells were treated with the extract at the final concentrations determined in FIG. 5a (62.5, 125, 250, and 500 μg/ml) on day 1, on day 4, and on day 7, three times in total. To accumulate A2E in cells, the cells were treated with A2E at the final concentration of 10 uM on day 2, on day 5, and on day 8, three times for 7 days, which were then irradiated with blue light (4.02 J/cm²), followed by culture for 24 hours. According to MTT assay method, DMEM containing 0.5 mg/ml of MTT was added to the cells, and light was blocked, followed by reaction in a 37° C. incubator for 4 hours. Upon completion of the reaction, the cells were fully dissolved in 1 ml of DMSO. Then, OD₅₄₀ was measured with ELISA microplate reader. Cell survival rate was presented as a percentage (%)value calculated from the ratio of the survived cells to the number of cells having A2E accumulated but not irradiated with blue light.

As a result, as shown in FIG. 5b, there was a statistically significant difference in the cell survival rate between the group having A2E accumulated but not irradiated with blue light (A2E) and the group having A2E accumulated and irradiated with blue light (negative control: A2E BL). The cell survival rate of the cells treated with the KIOST originated Spirulina maxima extract at the concentrations of 62.5, 125, 250, and 500 μg/ml was increased respectively by 0%, 10.8%, 7.0%, and 11.9% based on that of A2E BL as 100% (FIG. 5b).

What is claimed is:

1. A pharmaceutical composition for preventing and treating a retinal disease comprising Spirulina maxima extract as an active ingredient.

2. The pharmaceutical composition for preventing and treating a retinal disease according to claim 1, wherein the Spirulina maxima extract comprises one or more ingredients selected from the group consisting of Allophycocyanin (APC), R-phycoerythrin (R-PE), and C-phycocyanin (C-PC).

3. The pharmaceutical composition for preventing and treating a retinal disease according to claim 1, wherein the Spirulina maxima extract is extracted by centrifugal extraction, solvent extraction, or ultrasonic extraction.

4. The pharmaceutical composition for preventing and treating a retinal disease according to claim 1, wherein the Spirulina maxima extract exhibits the effect of inhibiting pyridinium bis-retinoid (A2E) oxidized by blue light.

5. The pharmaceutical composition for preventing and treating a retinal disease according to claim 1, wherein the retinal disease is selected from the group consisting of macular degeneration, glaucoma, Usher syndrome, Stargardt disease, Bardet-Biedl syndrome, Best disease, choroideremia, gyrate-atrophy, retinitis pigmentosa, macular degeneration, Leber congenital amaurosis (Leber's Hereditary Optic Neuropathy), BCM (blue-cone monochromacy), retinoschisis, ML (Malattia Leventinese), Oguchi disease, and Refsum disease.

6. The pharmaceutical composition for preventing and treating a retinal disease according to claim 1, wherein the Spirulina maxima extract characteristically inhibits retinal cell death.

7. A health functional food for preventing and improving conditions of a retinal disease comprising Spirulina maxima extract as an active ingredient.

8. The health functional food for preventing and improving conditions of a retinal disease according to claim 7, wherein the Spirulina maxima extract is extracted by centrifugal extraction, solvent extraction, or ultrasonic extraction.

9. A method for treating retinal disease containing the step of administering an effective dose of the Spirulina maxima extract to a subject having a retinal disease.

10. A method for treating retinal disease containing the step of administering an effective dose of one or at least two of the ingredients selected from the group consisting of Allophycocyanin, R-phycoerythrin, and C-phycocyanin to a subject having retinal disease.
